(19) United States
(12) Patent Application Publication
(10) Pub. No.: US 2022/0298481 A1
(43) Pub. Date: Sep. 22, 2022

(54) METHOD FOR PREPARING ZONAL LAYERED CHONDROCYTE SHEETS AND TREATING METHOD THEREOF

(71) Applicant: KAOHSIUNG MEDICAL UNIVERSITY, Kaohsiung City (TW)

(72) Inventor: YIN-CHUN TIEN, Kaohsiung City (TW)

(73) Assignee: KAOHSIUNG MEDICAL UNIVERSITY, Kaohsiung City (TW)

(21) Appl. No.: 17/697,935

(22) Filed: Mar. 18, 2022

Related U.S. Application Data
(60) Provisional application No. 63/163,058, filed on Mar. 19, 2021.

(51) Int. Cl. C12N 5/077; A61P 19/04; A61K 35/32; A61L 27/36; A61L 27/38

(52) U.S. Cl. CPC C12N 5/0655; A61P 19/04; A61K 35/32; A61L 27/3612; A61L 27/3654; A61L 27/3852; A61L 27/3817; C12N 2513/00; C12N 2533/30; A61L 2430/06

(57) ABSTRACT

A method for preparing zonal layered chondrocyte sheets, comprising the steps: (a) providing a cartilage sample from a subject; (b) isolating chondrocytes from the cartilage sample and then isolating superficial zone chondrocytes, middle zone chondrocytes and deep zone chondrocytes from the chondrocytes; (c) culturing the deep zone chondrocytes until reaching 100% cell confluence to form a deep zone chondrocyte sheet; (d) seeding the middle zone chondrocytes on the top of the cultured deep zone chondrocyte sheet from the step (c) and culturing the middle zone chondrocytes until reaching 100% cell confluence to form a middle zone chondrocyte sheet; and (e) seeding the superficial zone chondrocytes on the top of the cultured middle zone chondrocyte sheet from the step (d) and culturing the superficial zone chondrocytes until reaching 100% cell confluence to form a superficial zone chondrocyte sheet to obtain the zonal layered chondrocyte sheets.

### CROSS-REFERENCE TO RELATED APPLICATION

This application claims the benefit of U.S. Provisional Patent Application No. 63/163,058 filed on Mar. 19, 2021, which is incorporated by reference herein in its entirety.

This application contains a Sequence Listing in a computer readable form, the file name is 3831-KMU-SEQListing-ST25, created on Mar. 31, 2022, the size is 7 KB, which is incorporated herein by reference.

### FIELD OF THE INVENTION

The present invention relates to a method for preparing zonal layered chondrocyte sheets, which is characterized by isolating articular chondrocytes from a cartilage sample of a subject, then separating these chondrocytes into three groups of cell: superficial zone chondrocytes, middle zone chondrocytes and deep zone chondrocytes, culturing the above three kinds of chondrocytes respectively for cell proliferation, and then in order, constructing a deep zone chondrocyte sheet from the deep zone chondrocytes, seeding the middle zone chondrocytes on the top of the deep zone chondrocyte sheet to construct a middle zone chondrocyte sheet, and finally seeding the superficial zone chondrocytes to construct a superficial zone chondrocyte sheet, thereby building stratified sheets having a three-layer structure from bottom to top.

### DESCRIPTION OF PRIOR ART

Articular cartilage damage is hard to self-repair due to its avascular, a neural structural organization, and low cell-to-matrix ratio. Articular cartilage injuries often result in early onset of degenerative joint diseases. Up to now, attaining fully functional cartilage tissue repair still faces significant challenge. Autologous chondrocyte implantation (ACI) is currently a promising treatment strategy and the only FDA-approved cell-based therapy method for treating cartilage damage. Although ACI has been successfully to regenerate hyaline-like of neo-cartilage in some clinical cases when compared to the traditionally approach of microfracture. The microfracture approach is widely used because of their simplicity and low cost. However, this approach is only valid for small lesion, and provides relatively short-term functional improvement due to the formation of fibrocartilage rather than hyaline articular cartilage. With larger defect and/or more severe baseline symptoms, the ACI approach has been displayed to be more efficacious than microfracture in short term studies.

To consider of native articular cartilage and reconstruct the complex zonal organization is critical for designing the suitable strategy for functional tissue repair. The articular cartilage is composed of matrix containing plentiful collagen fibers and proteoglycans (PG), embedding chondrocytes which provide the synthesis of cartilage extracellular matrix (ECM). Articular cartilage divided into three zones including the superficial (SZ), middle (MZ) and deep zone (DZ) and characterized by different cell morphology and density, structural arrangement of the ECM, organizational complexity as well as biomechanical properties. The SZ (composition of the upper 10-15% of total cartilage) contains relatively high density and flattened chondrocytes which collagen fibrils are oriented parallel to the articular surface. Chondrocytes in the superficial zone produce GAG at a relatively low rate and secret a specific superficial zone protein (SZP) which play a role for smooth gliding motion during joint movement. The MZ (surface to 40-50% of total cartilage thickness) contains more rounded chondrocytes and the collagen fibers with random orientation. The DZ (30-40% of total cartilage thickness) compose of large chondrocytes in vertical columns and the collagen fibrils aligned perpendicularly to the articulating surface. Several markers exist in DZ such as the Notch-Delta signaling pathway and collagen type X which are expressed in the deepest layers of articular cartilage. The collagen fibrils are dominant the collagen type II (Col 2) and the aggrecan (Aggr), but also contain other minor collagen type IX (Col 9), and type XI, which are important in the regulation of fibril size, interfibril cross-linking, and interactions with the PGs. Due to the variations in the structural arrangement of collagen and proteoglycan lead to significant differences in biomechanical properties including the tensile, compressive and shear properties corresponded to the depth of the cartilage. In addition, the specific zonal structure of cartilage is also important in regulating the appropriate signaling to the different layers of cartilage tissue. For example, reserving the superficial surface of cartilage is not only important for cartilage integrity and lower friction on articular cartilage surface but also regulating the proliferative and metabolic activities of the deep zone chondrocytes. However, ACI strategies often implant mixed chondrocytes comprising SZ, MZ and DZ on a biomaterial scaffold, consequently, the regenerated cartilages constructed by cartilage tissues engineering in the past are often homogenous, have no stratified structure and lack of their native structure of SZ, MZ and DZ.

From these previous studies suggest that fabricate the stratified characterization of native articular cartilage may enhance the neo-cartilage mechanical properties and keep the long-term function of the regenerated cartilage. In recent years, many approaches try to use the scaffold- and matrix-free, scaffold/matrix-based and hybrid approaches to prepare zonal layered articular cartilage. In which, the simplest micro-mass pellet culture approach that pellets do develop a zonal organization of cells and matrix, with GAG content typically increasing from the outer surface to the center. However, this method is not suitable directly application for clinical treatment, as the pellets are small, the zonal variations are spherical rather than depth dependent, and is not able to restore a cartilage defect of >1 cm². The scaffold/matrix approach still has a problem about the control of scaffold pore-size and interconnectivity, the difficulty to seed cells into the scaffolds evenly (typically displayed a high density of cells at the periphery and low cell density in the center). In addition, the scaffold is a foreign material to the body and might induce undesired inflammation and immune response. Further, some researches try to use the hydrogel with gradients in stiffness or composition for seeding zonal specific chondrocytes to construct the stratified framework. However, this method often resulted in delamination caused by weak connection between the various layers. To address the issues, one feasible approach to overcome the described problem is the utilization of cell-sheet technology; this is due to cell sheets technology facilitates easily stacking of different zone chondrocytes. And its scaffold free property will not cause inflammation and eliminate the risk of an immune rejection. More recently, cell sheets had been applied in regenerative medicine, such as the regeneration of the myocardium, cornea, and renal cells. In addition, this technology also had been developed chondrocyte sheets in repair the articular cartilage defect. However, in the past, the chondrocyte sheets do not have a structure of SZ, MZ and DZ.

### SUMMARY OF THE INVENTION

The present invention provides a method for preparing zonal layered chondrocyte sheets, comprising the steps: (a) providing a cartilage sample from a subject; (b) isolating chondrocytes from the cartilage sample and then isolating superficial zone chondrocytes, middle zone chondrocytes and deep zone chondrocytes from the chondrocytes; (c) seeding the deep zone chondrocytes in a culture medium in a culture dish and culturing the deep zone chondrocytes until reaching 100% cell confluence to form a deep zone chondrocyte sheet; (d) seeding the middle zone chondrocytes on the top of the cultured deep zone chondrocyte sheet from the step (c) and culturing the middle zone chondrocytes until reaching 100% cell confluence to form a middle zone chondrocyte sheet; and (e) seeding the superficial zone chondrocytes on the top of the cultured middle zone chondrocyte sheet from the step (d) and culturing the superficial zone chondrocytes until reaching 100% cell confluence to form a superficial zone chondrocyte sheet to obtain the zonal layered chondrocyte sheets having the deep zone chondrocyte sheet, the middle zone chondrocyte sheet and the superficial zone chondrocyte sheet.

### DETAILED DESCRIPTION OF THE INVENTION

This present invention aims to use the cell sheet technology. The present invention isolates superficial zone chondrocytes, middle zone chondrocytes and deep zone chondrocytes and uses the cell sheet technology to prepare a regenerated cartilage with stratified structure.

To restitute the stratified architecture of native articular cartilage that has known as a pivotal factor required for recapitulating the biomechanical properties and obtaining long-term tissue integrity of the repaired articular cartilage. The present invention uses a discontinuous Percoll gradient centrifugation method for sorting of three kinds of chondrocytes comprising superficial zone chondrocytes, middle zone chondrocytes and deep zone chondrocytes from the chondrocytes. As shown in FIGS. 1A-1E, chondrocytes obtained from distal femur cartilage are separated into three layers of cells which contain morphological and phenotypic differences. The upper most fraction is derived largely from the deep zone of articular cartilage which contained the larger cell and high concentration of proteoglycan. In contrast, the cells of lowest fraction are derived from superficial zone which the cell is smallest, relatively low concentration of proteoglycan and divide more slowly than those from the middle zone, and cells from the superficial zone. Further, detecting ECM markers and secreted protein associated with different cartilage zones, the present invention demonstrates the chondrogenic properties of the density gradient-sorted subpopulations, respectively. Significantly higher expression of Aggrecan and Col-2a1 is found in middle/upper most fraction, relative to the lowest fraction that represent the two fraction is the middle zone and deep zone of articular cartilage. These results are consistent with the previously studies that using dissection methods and cell size-based inertial spiral microchannel technique for the separation of the superficial, middle and deep zone chondrocytes from the full thickness (FT) cartilage blocks. Conversely, the superficial zone PRG4 is found to be specifically expressed in high level in the lowest fraction that also demonstrated mostly of cells are from superficial zone.

From the in vitro experiments revealed that the cartilage formation of the stratified cell sheets that is functional superior than that in traditional heterogeneous sheets (non-layered). These research results find the following advantages: (1) The present invention has fabricated the three layers cell sheet (SZ, MZ, DZ) of articular chondrocytes and analyzed the chondrogenic marker by real time PCR and found that the col-2a1 and aggrecan mRNA are significantly increased in stratified cell sheet compared to heterogeneous cell sheet, in contrast, the MMP13 mRNA expression level in stratified cell sheet is less than in heterogeneous cell sheet. In addition, the cell proliferation rate of stratified sheets is superior when in comparison with the heterogeneous sheets. (2) Stratified sheets secreted significantly higher concentrations of TIMP-1 and TIMP-3, and lower concentrations of matrix metalloproteinases (MMP)-3 and MMP13 than heterogeneous sheets. (3) Less pro-inflammatory cytokine such as IL-6, IL-8 and TNF-α produced in stratified sheets than in heterogeneous sheets (FIGS. 1A-1E). It is currently known that chondrocytes are capable of producing inflammatory cytokines that negatively impact the tissue via autocrine and paracrine pathway. From several studies demonstrate that the IL-1β expression of implanted chondrocyte significantly influence the clinical outcome after autologous chondrocyte transplantation. Hence, the ability to regulate expression of principal cytokines such as IL-1β and TNF-α within the cell sheets may lead to an improvement of ACI performance. (4) Histological evaluation shows that stratified sheets produce more proteoglycan deposition than heterogeneous sheets by assessing with Alcian blue staining and detecting GAG content. (5) Immunofluorescence and Western blot analysis shows weak staining for a disintegrin and metalloproteinase with thrombospondin motifs (ADAMTS)-4, ADAMTS-5 in stratified sheets and better staining for Col-2a1 and Aggrecan.

In addition, the present invention uses the zonal layered chondrocyte sheets in repairing cartilage defect using a porcine defect model, in comparison to the use of heterogeneous chondrocytes sheet (non-layered). From histological scoring shows that implantation of Percoll density separated zonal chondrocytes as tri-layered sheets, the neo-cartilage displayed hyaline like and a native cartilage-characteristic zonal structure after implantation of 12 weeks (FIGS. 2 and 3). Besides, the yielded cartilage of better quality, with significant improvement compared with implantation of un-layered chondrocytes or control group.

As used herein, the term “a” or “an” are employed to describe elements and components of the present invention. This is done merely for convenience and to give a general sense of the present invention. This description should be read to include one or at least one and the singular also includes the plural unless it is obvious that it is meant otherwise.

The present invention provides a method for preparing zonal layered chondrocyte sheets, comprising the steps: (a) providing a cartilage sample from a subject; (b) isolating chondrocytes from the cartilage sample and then isolating superficial zone chondrocytes, middle zone chondrocytes and deep zone chondrocytes from the chondrocytes; (c) seeding the deep zone chondrocytes in a culture medium in a culture dish and culturing the deep zone chondrocytes until reaching 90-100% cell confluence to form a deep zone chondrocyte sheet; (d) seeding the middle zone chondrocytes on the top of the cultured deep zone chondrocyte sheet from the step (c) and culturing the middle zone chondrocytes until reaching 90-100% cell confluence to form a middle zone chondrocyte sheet; and (e) seeding the superficial zone chondrocytes on the top of the cultured middle zone chondrocyte sheet from the step (d) and culturing the superficial zone chondrocytes until reaching 90-100% cell confluence to form a superficial zone chondrocyte sheet for obtaining the zonal layered chondrocyte sheets having the deep zone chondrocyte sheet, the middle zone chondrocyte sheet and the superficial zone chondrocyte sheet.

In one embodiment, the cartilage sample is an articular cartilage sample. In a preferred embodiment, the cartilage sample is a cartilage tissue. In a more preferred embodiment, the cartilage sample is an articular cartilage tissue.

As used herein, the term “subject” refers to an animal. In a preferred embodiment, the subject refers to a mammal. In a more preferred embodiment, the subject refers to a human.

The present invention uses the density gradient centrifugation to isolate the superficial zone chondrocytes, middle zone chondrocytes and deep zone chondrocytes from the chondrocytes. In another embodiment, the isolating method in the step (b) comprises using a technique of cell separation by density gradient centrifugation. In a preferred embodiment, the range of the density gradient comprises 1.015 to 1.07 g/ml. In a more preferred embodiment, the rate of the density gradient centrifugation is 400×g for 20 to 30 min.

In the present invention, the superficial zone chondrocytes, middle zone chondrocytes and deep zone chondrocytes are cultured respectively after isolating three kinds of chondrocytes. In one embodiment, the culture medium comprises DMEM/F12, fetal bovine serum, ascorbic acid, and antibiotics. The purpose of the culturing is used for cell proliferation to achieve the cell number for seeding. In one embodiment, the step (b) comprises culturing the superficial zone chondrocytes, the middle zone chondrocytes and the deep zone chondrocytes respectively after isolating three kinds of chondrocytes.

In one embodiment, the cell density of the superficial zone chondrocytes, the middle zone chondrocytes and the deep zone chondrocytes for seeding ranges from 1×10⁴ to 5×10⁴ cells/cm².

In one embodiment, the deep zone chondrocytes are cultured for 3 to 5 days for reaching 90-100% cell confluence to form the deep zone chondrocyte sheet.

In another embodiment, the middle zone chondrocytes are cultured for 3 to 5 days for reaching 90-100% cell confluence to form the middle zone chondrocyte sheet.

After the superficial zone chondrocytes are seeded on the cultured middle zone chondrocyte sheet, the superficial zone chondrocytes are cultured for 3 to 5 days for reaching 90-100% cell confluence to form the superficial zone chondrocyte sheet.

In another embodiment, the deep zone chondrocytes, the middle zone chondrocytes and the superficial zone chondrocytes are cultured until reaching 95-100% cell confluence. In a preferred embodiment, the deep zone chondrocytes, the middle zone chondrocytes and the superficial zone chondrocytes are cultured until reaching 100% cell confluence.

During the preparing process of the zonal layered chondrocyte sheets, the deep zone chondrocytes, the middle zone chondrocytes and the superficial zone chondrocytes secrete cell factors to form extracellular matrix, respectively. Therefore, the final product of the zonal layered chondrocyte sheets comprises an extracellular matrix with different zone characteristics.

In the present invention, the zonal layered chondrocyte sheets also need to be cultured for additional 1 to 3 weeks after the superficial zone chondrocyte sheet is formed. During the preparing process of the zonal layered chondrocyte sheets, the deep zone chondrocytes, the middle zone chondrocytes and the superficial zone chondrocytes are continuously cultured. In one embodiment, the culture time of the zonal layered chondrocyte sheets after seeding the superficial zone chondrocytes ranges from 1 to 4 weeks. In a preferred embodiment, the culture time of the zonal layered chondrocyte sheets after seeding the superficial zone chondrocytes ranges from 1 to 3 weeks.

In addition, the culture medium for culturing the zonal layered chondrocyte sheets further is added suramin. The suramin can promote chondrocyte differentiation by enhancing the Col-2a and Aggrecan expression and lowering Col-1a synthesis. During the culturing of the zonal layered chondrocyte sheets, the suramin remarkably suppresses the expression of matrix destruction proteases and inflammatory mediators, meanwhile enhances the production of cartilage anabolic factors in interleukin-1β-induced (IL-1β) chondrocyte sheet. In one embodiment, the culture medium for culturing the zonal layered chondrocyte sheets in the step (e) comprises suramin. In addition, in the step (e), the zonal layered chondrocyte sheets are isolated from the culture medium for obtaining the zonal layered chondrocyte sheets having the deep zone chondrocyte sheet, the middle zone chondrocyte sheet and the superficial zone chondrocyte sheet. In another embodiment, the step (e) further comprising isolating the zonal layered chondrocyte sheets from the culture medium.

Base on the preparation method of the present invention, the arrangement of the three sheets from bottom to top is as follows: the deep zone chondrocyte sheet, the middle zone chondrocyte sheet and the superficial zone chondrocyte sheet. Thus, the zonal layered chondrocyte sheets of the present invention are a complex of the cartilage sheets comprising the deep zone chondrocyte sheet (comprising the extracellular matrix of the chondrocyte with the characteristics of the deep zone chondrocyte) the middle zone chondrocyte sheet (comprising the extracellular matrix of the chondrocyte with the characteristics of the middle zone chondrocyte), and the superficial zone chondrocyte sheet (comprising the extracellular matrix of the chondrocyte with the characteristics of the superficial zone chondrocyte). The zonal layered chondrocyte sheets having three-layer structures prepared by the present invention are similar with native cartilage.

The present invention also provides a method for treating cartilage defects comprising administering a composition to a cartilage defect site of a subject, wherein the composition comprises zonal layered chondrocyte sheets. The zonal layered chondrocyte sheets are prepared by the method of the present invention.

The term “treat” refers to any improvements of a disease or illness (also refers to inhibition of the disease or amelioration of the appearance, extent or severity of at least one of its clinical symptoms).

As used herein, the term “cartilage defects” includes, but is not limited to, cartilage degeneration or diseases of cartilage defects/wear caused by age, gene mutations or damages cause by external force. The cartilage widely exists in the articular surface of a bone, costal cartilage, trachea, pinna, lumbar discs. In one embodiment, the cartilage defects comprise articular cartilage defects.

The preferred route of administration of the composition of the present invention to the subject comprises intraarticular administration.

The present invention further provides a composition comprises zonal layered chondrocyte sheets. The zonal layered chondrocyte sheets are prepared by the method of the present invention.

In addition, the present invention provides a use of a composition in the preparation of a pharmaceutical composition for treating cartilage defects, wherein the composition comprises zonal layered chondrocyte sheets. The zonal layered chondrocyte sheets are prepared by the method of the present invention. In one embodiment, the zonal layered chondrocyte sheets comprise an extracellular matrix which exists the zonal characteristics.

In another embodiment, the cartilage defects comprise articular cartilage defects. In a preferred embodiment, the route of administration of the composition comprises intraarticular administration.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E show comparison the pro-inflammatory cytokine expression in heterogeneous (non-layered) articular chondrocytes (ACs) sheets and stratified sheets. To determine the proinflammatory gene expression levels, the total RNA was extract at 3 weeks after triple layer sheets were fabricated. The mRNA levels of IL1-β (FIG. 1A), TNF-α (FIG. 1B), MIF (FIG. 1C), IL-6 (FIG. 1D) and IL-8 (FIG. 1E) were measured by qRT-PCR. Data are statistically significantly different if *p<0.05, ***p<0.001.

FIGS. 2A-2F show macroscopic and histological observation of repaired cartilage. FIG. 2A shows the photographs represent of porcine knee articular defect healing in the control, non-layered and layered cell sheets implantation groups at 12 weeks after surgery. The red circles indicate the original defect margin. FIG. 2B shows ICRS macroscopic assessment scores of repaired cartilage at 12 weeks. Data are presented as mean±SD (n=5, *P<0.05, **P<0.01 and ***P<0.001). FIG. 2C shows H&E staining. Boxes in left panels represent magnified area shown in right panels. FIG. 2D shows the staining results of Safranin 0/Fast green in the control, non-layered and layered cell sheets implantation groups at 12 weeks after surgery. FIG. 2E shows the staining results of Alcian bluein the control, non-layered and layered cell sheets implantation groups at 12 weeks after surgery. Scale=100 μM. FIG. 2F shows the sections were scored with Mankin score in small (left) and larger magnification (right image, #). Arrowheads indicate the lacunae.

FIGS. 3A-3C show immunohistochemical staining of regenerated cartilages at 12 weeks. FIG. 3A shows the representative images of immunohistochemical staining for Col-2a1, Col-10a1 and Aggrecan. The blue rectangle indicates the original defect margin and magnified as larger image at right side. And the quantitative analysis of the IOD for Col-2a1 staining (FIG. 3B), Aggrecan staining (FIG. 3C) and Col-10a1 staining (FIG. 3D) of the three groups at 12 weeks with and without treatment with cell sheet transplantation. Data are presented as mean±SD (n=6, *P<0.05, **P<0.01 and ***P<0.001).

### EXAMPLES

The present invention may be implemented in many different forms and should not be construed as limited to the examples set forth herein. The described examples are not limited to the scope of the present invention as described in the claims.

Materials and Methods

1. Cell Preparation and Cell Sheets Construction

All animal procedures were reviewed and approved by Institutional Review Board (IRB). The 5-8-month-old porcine articular cartilage of distal femur were harvested. The chondral defect measuring 8 mm in diameter and 5 mm deep was made in the unweight bearing area of femoral condyle using a biopsy punch. The harvest articular cartilage blocks were cut into small pieces by a scalpel and incubated in 0.1% (w/v) trypsin in PBS under gentle agitation for 20 min at 37° C. The trypsin was removed and the pieces were washed with culture medium and digested overnight with 0.01% (w/v) (0.166 U/ml) collagenase P (Boehringer/Roche Mannheim, Germany) in medium in the presence of 10% fetal calf serum (FCS) at 37° C. under agitation. The articular cartilage pieces were then digested in supplemented DMEM/F12 containing 1.5 mg/ml collagenase type II and 5% FBS at 37° C. for 10 hr. After the 10 h enzymatic digestion of the extracellular matrix, the freed cells were separated from tissue debris by filtration through a 70-μm nylon cell strainer (Becton Dickinson, Franklin Lakes, N.J.) and collected from the filtrate by centrifugation at 150×g for 5 min. Cells were then washed in 1×PBS twice and resuspended in 1 ml of DMEM/F12 medium. Separation of various zonal chondrocytes was according to Byoung et al report (Min B H, Kim H J, Lim H, Park S R.

Characterization of subpopulated articular chondrocytes separated by Percoll density gradient. In vitro cellular & developmental biology Animal. 2002; 38(1):35-40) with some modification. In brief, articular cartilage cells were layered on a discontinuous isotonic Percoll (GE Healthcare) density gradient prepared by weight (densities of 1.015-1.07 g/ml) and centrifuged at 400×g for 20-30 min in a swinging bucket rotor. To fabricate the heterogeneous or stratified cell sheets, articular or deep zone chondrocytes were harvest according to the method described above and seeded on 6 well culture dish at 1×10⁴-5×10⁴ cells/cm² in DMEM/F12 supplemented with 10% fetal bovine serum (FBS; GIBCO, NY, USA), 100 μg/ml ascorbic acid, and 1% antibiotics-antimycotic (GIBCO, NY, USA) at 37° C. in an atmosphere of 5% CO₂ and 95% air. Continuous culture about 3-5 days until the first layer chondrocyte reaching confluent, the second layer of chondrocytes (including heterogeneous chondrocyte and middle zone chondrocytes) were seeding on the first layer and continuous cultured about 3-5 days until 100% confluent. Then the third layer of chondrocytes (including heterogeneous chondrocyte and superficial zone chondrocytes) were subsequently seeded on the second layer and continuous culture for additional 1-3 weeks. In addition, the component of the culture dish could be further added Suramin during the additional 1-3 weeks. Three weeks later, a thin film formed in the cell culture dish, which was found containing a three layered chondrocytes and extracellular matrix (ECM) under inverted microscope. The sheets were collected onto a polyvinylidene difluoride (PVDF) membrane according the method reported by Yamato et al (Yamato M, Utsumi M, Kushida A, et al. Tissue engineering. 2001; 7(4):473-480). The heterogeneous sheets (non-layered) and stratified sheets (layered) were harvested and processed for biochemical, histological and immunofluorescence evaluation.

2. Cell Proliferation and Viability

For detecting the cell proliferation rate, the cell number of heterogeneous sheets (non-layer) and stratified sheets (layer) were directed counted. The sheets were digested with TrypLE Express for 30 min at 37° C. followed by incubation with 0.25 mg/mL Collagenase-P for 30 min at 37° C. The dispersed cells were collected and counted using counting chamber. The cell viability was determined by MTT assay.

3. Gene Expression of Cell Sheets

Total RNA of chondrocyte sheets was extracted using TRIzol (Invitrogen). 2 μg of purified total RNA was reverse-transcribed by the Thermo Scientific Maxima First Strand cDNA Synthesis Kit (ThermoFisher) according to the manufacturer's instructions. Briefly, the solution was incubated at 65° C. for 5 min, it was mixed with first-strand buffer, DTT, and RNaseOUT in a final volume of 20 μL. Then, the solution was incubated at 42° C. for 60 min and then at 70° C. for 15 min to inactivate the reverse transcriptase activity. Real-time PCR was conducted using the SYBR Green PCR Master Mix (Qiagen) and was processed on a LightCycler PCR and detection system (Roche Diagnostics). Each reaction (20 μl) was run in duplicate and contained 1 μl of cDNA template along with the following primer sequences:

col-2a1, forward
(ACTCCTGGCACGGATGGTC)
and
reverse
(CTTTCTCACCAACATCGCCC);
aggrecan, forward
(CCCAACCAGCCTGACAACTT)
and
reverse
(CCTTCTCGTGCCAGATCATCA);
col-10a1, forward
(TGAACTTGGTTCATGGAGTGTTTTA)
and
reverse
(TGCCTTGGTGTTGGATGGT);
gapdh, forward
(TCACGACCATGGAGAAGGCT)
and
reverse
(CAGGAGGCATTGCTGATGATC);
col-1a1, forward
(CTGGTACGGCGAGAGCATGACC)
and
reverse
(GGAGGAGCAGGGCCTTCTTGAG);
sox5, forward
(GGCCAAGCAGCAGCAAGAACAG)
and
reverse
(AGCTGAAGCCTGGAGGAAGGAG);
sox6, forward
(CAGCCCTGTCAGTCTGCCTAACA)
and
reverse
(GCATCTTCCGAGCCTCCTGAATAGC);
sox9, forward
(GGCAATCCCAGGGTCCACCAAC)
and
reverse
(TGGTCGAACTCGTTGACGTCGAAG);
mmp13, forward
(ACCCAGGAGCCCTCATGTTTCC)
and
reverse
(CAGGGTTTCTCCTCGGAGACTG);
runx2; forward
(CCAGACCAGCAGCACTCCATAC)
and
reverse
(GGGAACTGCTGTGGCTTCCATC);
prg4 forward
(CTCCCAAGGAGCAGCTTCTAC)
and
reverse
(GGTGGTGGGAGCTGGTTCCTTG);
pcna, forward
(GCGCCTGGTCCAGGGC)
and
reverse
(TCACGCCCATGGCCAAATTGC);
IL-1β, forward
(GTACATGGTTGCTGCCTGAA)
and
reverse
(CTAGTGTGCCATGGTTTCCA);
IL-6, forward
(GGCAGAAAACAACCTGAACC)
and
reverse
(GTGGTGGCTTTGTCTGGATT);
IL-8, forward
(TAGGACCAGAGCCAGGAAGA)
and
reverse
(CAGTGGGGTCCACTCTCAAT);
TNFα, forward
(ACTGCACTTCGAGGTTATCG)
and
reverse
(GCTGGTTGTCTTTCAGCTTC);
MIF, forward
(CGTGCGCCCTTTGCAGTCTG)
and
reverse
(TGGCCGCGTTCATGTCGTAG).

Cycling parameters were 95° C. for 15 min to activate DNA polymerase, followed by 40 cycles of 95° C. for 15 s, 60° C. for 20 s, and 72° C. for 30 s. Melting curves were generated at the end of the reaction. Threshold cycles (Cₜ) for each gene tested were normalized to the housekeeping GAPDH gene value (ΔCₜ) and every experimental sample was referred to its control (ΔΔCₜ). Fold change values were expressed as 2^{-ΔΔ}Cₜ.

4. Total Glycosaminoglycans (GAG) Quantification

Total sulfated GAG content was determined by using 1,9-dimethylmethylene blue (DMMB; Polysciences). Chondroitin sulfate C from shark cartilage was used as a standard. Briefly, 100 μL of the digested sample was combined with 1 ml dimethylmethylene blue dye solution, and the absorbance was immediately measured at 656 nm. DNA was measured using Hoechst 33258 dye. Briefly, 10 μL of the digested sample was combined with 200 μL Hoechst dye solution (0.7 μg/mL) Fluorescence measurements were taken with an excitation wavelength of 340 nm and emission wavelength of 465 nm. A standard curve was obtained from calf thymus DNA. GAG content was normalized to the amount of DNA measured per sample and expressed as μg GAG/μg DNA.

5. Measurement of Humoral Factors

A heterogeneous sheets and stratified sheets were cultured for 72 h in 3 mL of DMEM/F12 supplemented with 1% FBS and 1% AB. Supernatants were collected and centrifuged at 12,000 g for 10 min to remove cell debris. The concentrations of transforming growth factor beta-1 (TGF-β1), tissue inhibitor of metalloproteinases-3 (TIMP3), tissue inhibitor of metalloproteinases-1 (TIMP1), matrix metalloproteinase-3 (MMP3), matrix metalloproteinase-13 (MMP13) were measured using enzyme-linked immunosorbent assay (ELISA) kits. The signal detected for blank medium containing 1% FBS was subtracted to adjust for proteins contained in FBS. Measurements were repeated at least twice for each donor, and averages were used.

6. Immunofluorescence Assay

Frozen sections of triple-layered cell sheets were fixed and frozen by using OCT compound. Cell sheets were incubated with Col-2a1 primary antibodies (Proteintech, 15943-1-AP, 1:100 dilution), Aggrecan (Proteitech, 13880-1-AP), MMP3 (Proteitech 66338-1-Ig), MMP-13 (Proteintech, 18165-1-AP), ADAMTS-4 (ABclonal, A2525) ADAMTS-5 (ABclonal, A2836) and a secondary antibody (LEADGENE® Goat anti-Rabbit IgG (H+L)-TAMRA and LEADGENE®Goat anti-Mouse IgG (H+L)-FAM). The cell nuclei were stained with 4′-6-diamidino-2-phenylindole (DAPI). The samples were then observed and photographed observed under a high-quality fluorescence microscope.

7. Alcian Blue Staining

ACs sheets and stratified sheets will be harvested after culture and then be embedded and frozen in optimal cutting temperature compound. Then, sections 5 μm thick will be stained for proteoglycans with Alcian blue using standard methods.

8. Transplantation of Chondrocyte Sheets

After the cell sheets were prepared. The cell sheets were autologous implant into the same porcine. Before the surgery of implantation, 0.2 mg/kg dormicum and 40 μg/kg medetomidine will be given intramuscularly. Inhalation anesthesia will be used during the operation with a combination of isoflurane, dinitrogen monoxide, and oxygen. A chondral defect measuring 8 mm in diameter and 5 mm deep will be made in the area of the animal's medial femoral condyle using a biopsy punch and the full thickness cartilage damaged will be covered with or without chondrocyte sheet. This will be performed in the knees of 6 minipigs (aged 7 months) in the transplantation group. Six porcines will be divided into three groups. 1st group (n=6): receive a femur defect and fill with three-layered stratified sheet, the 2nd group (n=6): receive a femur defect without filling cells. The 3rd group (n=6): receive a femur defect and fill with heterogeneous ACs sheets. The cartilage was harvested after 12 weeks, fixed in 4% paraformaldehyde for 1 week, and decalcified for 1 month. Next, the specimens were embedded in paraffin, cut into 5 μm sections, and stained with safranin-O, Alcian blue.

9. HE Stain and Immunohistochemical Examination

The harvest cartilage pieces were fixed in 4% paraformaldehyde, dehydrated in a graded ethanol and then embedded in paraffin. Specimens were stained with Hematoxylin and Eosin (H&E), safranin-O and Alcian blue. Immunohistological analysis was also performed. Col-2a1 primary antibodies (Proteintech, 15943-1-AP, 1:100 dilution), Aggrecan (Proteitech, 13880-1-AP), Col-10a1 (Abcam, ab49945) and the secondary antibody (DAKO) were successively subjected to immunohistological assay. The samples were then observed and photographed under a high-quality microscope.

10. Histological Grading Score for Assessment of Cartilage Repair

Tissue sections were evaluated using the histological grading score of Mankin (Mankin H J, Dorfman H, Lippiello L, Zarins A. Biochemical and metabolic abnormalities in articular cartilage from osteo-arthritic human hips. II. Correlation of morphology with biochemical and metabolic data. The Journal of bone and joint surgery American volume. 1971; 53(3):523-537), modified as described previously (Sakakibara Y, Miura T, Iwata H, et al. Effect of high-molecular-weight sodium hyaluronate on immobilized rabbit knee. Clinical orthopaedics and related research. 1994(299):282-292). The total score ranges from 0 to 14 and includes scores from four categories: cartilage structure, cellular abnormality, matrix staining, and tidemark integrity. Cartilage structure was graded from 0 (normal tissue) to 6 (cartilaginous tissue with complete disorganization). Cellular abnormality was graded from 0 (normal tissue) to 3 (hypocellularity). Matrix staining (with safranin-O) was graded from 0 (normal tissue or slightly decreased staining) to 4 (no staining). Tidemark integrity was graded from 0 (intact) to 1 (destroy). Based on the sum of the scores, each section was ranked as one of four histological grades: normal, 0-2; mild, 3-6; moderate, 7-10; or severe, 11-14.

11. Macroscopic Evaluation

The porcine in each group were sacrificed after transplantation of cell sheets at 12 weeks, and the cartilage were harvested. The defect sites were photographed and scored by using the International Cartilage Repair Society (ICRS) scoring system. The total score ranges from 0 to 12 and includes scores from three categories: degree of defect repair, integration to border zone, and macroscopic appearance. Degree of defect repair was graded from 0 (no repair) to 4 (in level with surrounding cartilage). Integration to border zone was graded from 0 (no contact to ¼ of graft integrated with surrounding cartilage) to 4 (complete integrated with surrounding cartilage). Macroscopic appearance graded from 0 (total degeneration of graft area) to 4 (intact smooth surface).

Results

Separates zonal articular chondrocytes and evaluate functional property of superficial zone (SZ), middle zone (MZ), and deep zone (DZ) chondrocytes

The full thickness of femur articular cartilage of 5 months old porcine was excised and collected. There are three distinct zones, namely SZ, MZ and DZ in the femur articular cartilage. And SZ, MZ and DZ constitute the top 10-15%, the middle 40-50%, and the deep 30-40% of the total cartilage thickness. Those chondrocytes in the three zones of epiphyseal cartilage are different in their cell size (data not shown). Based on the physical properties, chondrocytes derived from the three zones were fractionated by discontinuous Percoll gradient and the densities were settled at 1.015-1.07 g/ml. After centrifugation, the SZ, MZ, and DZ chondrocytes that had different buoyancies were distributed in different Percoll density layers. Considering that DZ has lower cell density as compared to MZ and SZ, hence the present invention collected the largest cells in the most upper fraction as DZ, the cells in the middle layer that are MZ chondrocytes, and the cells in lowest layer that are smallest size chondrocytes as SZ (data not shown). To verify whether the density gradient strategy actually separated the chondrocytes from different zones, the present invention further analyzed the mRNA expression levels of col-2a1, aggrecan, col-1a1, col-10a1, sox5, sox6, sox9, mmp13, runx2 and prg4 (data not shown). The data showed the significant expression of genes important for chondrocyte differentiation and cartilage maintenance, including col-2a1, aggrecan, sox5, sox6, and sox9 was higher in MZ. In contrast, the expression of col-10a1, mmp13 and runx2 was higher in DZ. Prg4 was found to be highest expression in SZ. Furthermore, SZ with less cell growth rate, cell viability and synthesized cartilage matrix by lower levels of glycosaminoglycans and proteoglycan than MZ and DZ.

Stratified chondrocytes cell sheet promotes the cell viability, cell proliferation and the expression of chondrogenic markers

For comparing the repair quality for cartilage defects, the zonal chondrocytes including SZ, MZ and DZ subpopulations were fabricated the tri-layer cell sheets (SZ, MZ and DZ are stacked in order from top to bottom, stratified articular chondrocytes sheets) versus traditional cell sheets made up of mixed chondrocytes (heterogeneous articular chondrocytes sheets) in vitro culture, after an additional 3 weeks expansion, the cells were harvested and counted (data not shown), the results showed that the number of cells in stratified cell sheets was significantly higher compare to heterogeneous cell sheets. In addition, transcriptional analysis of proliferating cell nuclear antigen (PCNA) showed a proportionate 2.5-fold elevated in stratified cell sheets group (data not shown). The average live cell percentage was slightly increased in the stratified cell sheets by using MTT assay (data not shown). From early studies has found that many marker gene expression of implant chondrocytes such as the Col-1a1, Col-2a1, Aggrecan, interleukin-1β (IL-1β), and bone sialoprotein-2 (BSP-2) influence the clinical outcome of ACI. To compare the cartilage forming capacity of two kind of cell sheets, the present invention analyzed the chondrogenic marker by real time PCR and found the col-2a1 and aggrecan mRNA were obviously increased in stratified cell sheet compared to heterogeneous cell sheet (col-2a1, Stratified ACs sheets vs heterogeneous ACs sheets, 4.8 fold; aggrecan, Stratified ACs sheets vs heterogeneous ACs sheets, 30 fold), in contrast, the mmp13 mRNA expression level in stratified cell sheet is less than in heterogeneous cell sheet (Stratified ACs sheets vs. heterogeneous ACs sheets, 0.8 fold). (data not shown)

Stratified sheets secreted lower concentrations of ECM destruction enzyme than heterogeneous sheets

In order to investigate the TGF-β, MMP-3, MMP-13, TIMP-1 and TIMP-3 protein levels produced by heterogeneous ACs sheets and stratified ACs sheets, supernatants of cell sheets cultures were collected and subjected to ELISA. The concentrations of humoral cytokines secreted by heterogeneous ACs sheet and stratified ACs sheets are summarized (data not shown). The stratified ACs sheets produced higher concentrations of TIMP-3 (stratified sheets 6100 to 6200 pg/mL; heterogeneous sheets 5320 to 5470 pg/mL), TIMP-1 (stratified sheets 31 to 33 ng/mL; heterogeneous sheets 22 to 23 ng/mL) (data not shown). And heterogeneous ACs sheets produced higher concentrations of MMP3 (stratified sheets 7 to 8 ng/mL; heterogeneous sheets 22 to 26 ng/mL) (data not shown), MMP-13 (stratified sheets 260 to 275 ng/mL; heterogeneous sheets 320 to 340 ng/mL) (data not shown). The concentrations no differed significantly between heterogeneous sheets and stratified sheets for TGF-β1 (data not shown).

Pro-inflammatory cytokines gene expression in stratified sheets was less than heterogeneous sheets

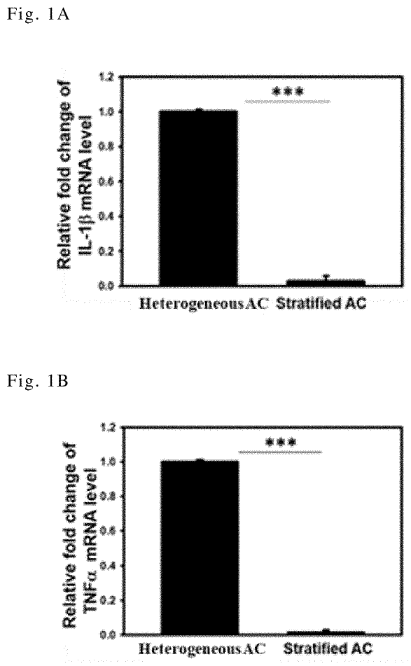

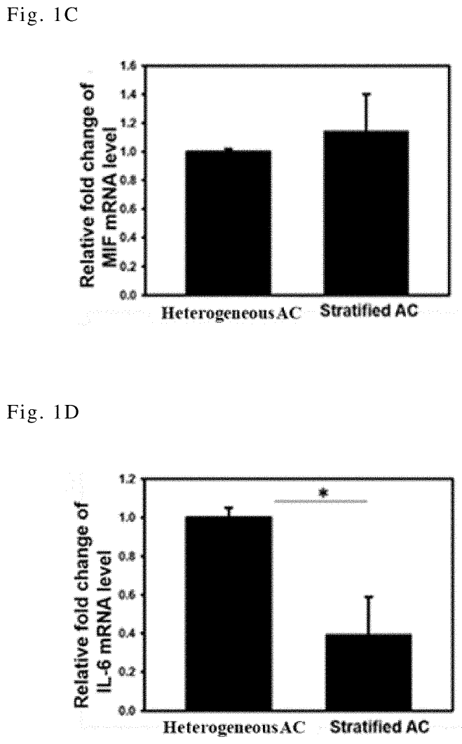

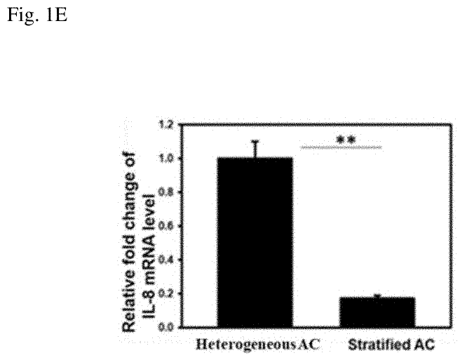

From previous reports has found that expression level of pro-inflammatory cytokines such as the IL-1β and TNF-α in the transplant has negative effect on clinical outcomes after ACI treatment. Hence, the present invention detected the pro-inflammatory cytokines gene expression including the IL1-β, TNF-α, IL-6, IL-8 and MIF in stratified sheets and heterogeneous sheets by qRT-PCR. As shown in FIGS. 1A-1E, the gene expression of IL1-β, TNF-α, IL-6, IL-8 in the stratified sheets were obvious less than heterogeneous sheets (IL1-β, heterogeneous sheets vs stratified sheets, 0.03 fold in stratified sheets; TNF-α, 0.01 fold in stratified sheets; IL-6, 0.4 fold in stratified sheets; IL-8, 0.2 fold in stratified sheets).

Comparison of matrix production ability and immunohistochemical analyses of stratified and heterogeneous ACs sheet

In order to investigate the chondrogenic properties on the stratified and heterogeneous ACs sheet, the Western blot, Alcian blue staining and immunofluorescence were performed. The expression of Col-2 which is cartilage-specific matrix collagens, was significantly greater in the stratified ACs sheets (data not shown). By contrast, the expression of the proteases MMP3 and MMP13 was low in the stratified ACs sheets (data not shown). The expression of ADAMTS-5, an extracellular protease enzyme that is closely participated in the progression of cartilage destruction, was also low in the stratified ACs sheets (data not shown). The Alcian blue staining demonstrated the deeper blue staining was shown in the stratified ACs sheet than in heterogeneous ACs sheets. That indicated the total proteoglycan deposition, an indicator of the ability to produce extracellular matrix was higher in stratified ACs sheet (data not shown), Further, immunofluorescence analysis showed higher staining for Col-2a1 and Aggrecan in the stratified chondrocytes cell sheet than in heterogeneous sheets. In contrast, less staining for MMP-3, MMP-13, ADAMTS-4 and ADAMTS-5 in the stratified chondrocytes cell sheet than in heterogeneous sheets (data not shown).

In Vivo Repair Evaluation by Gross Appearance and Histology

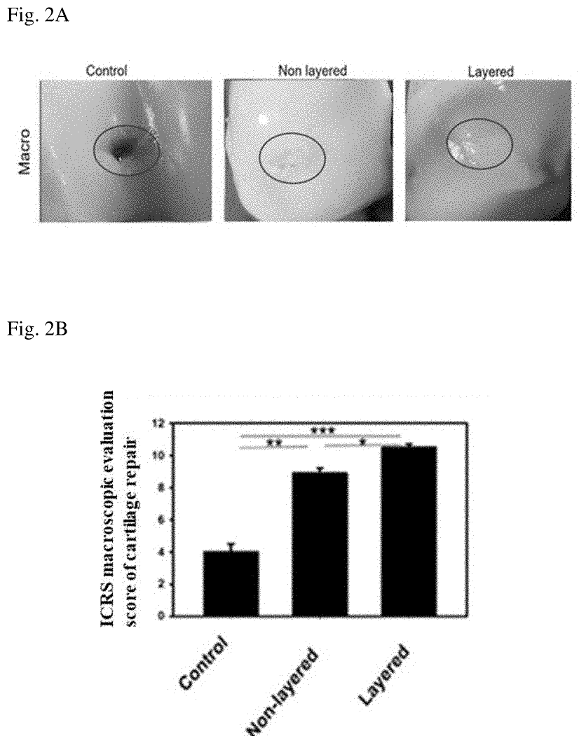

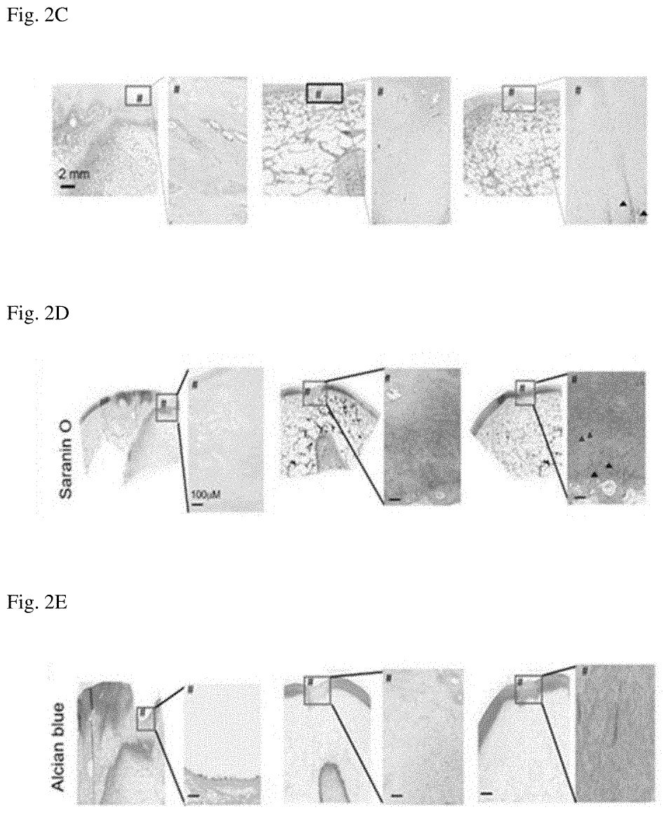

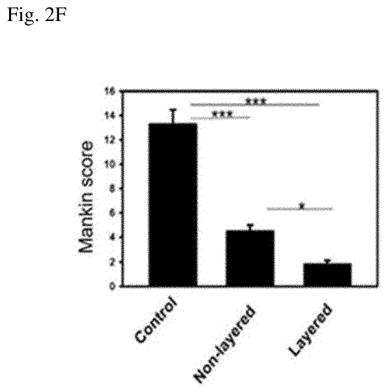

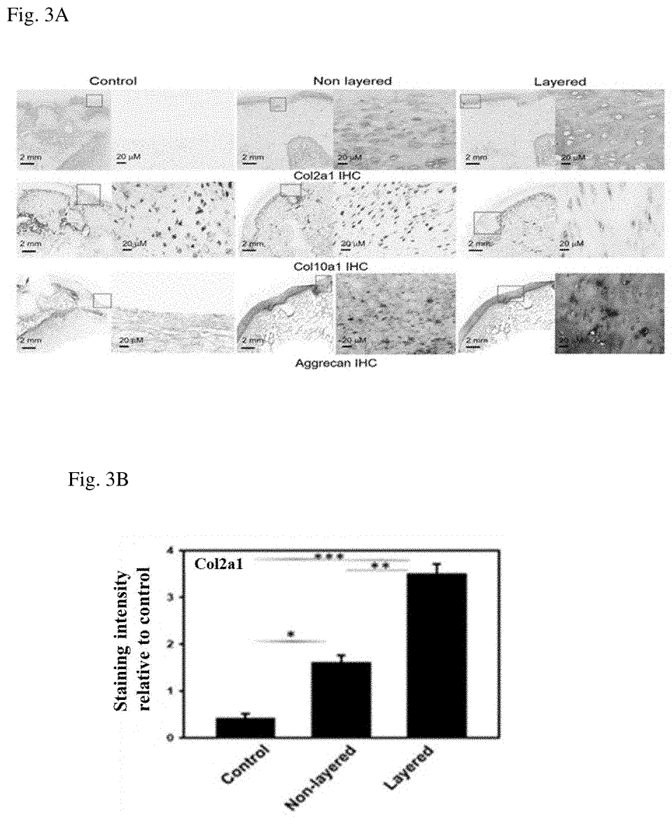

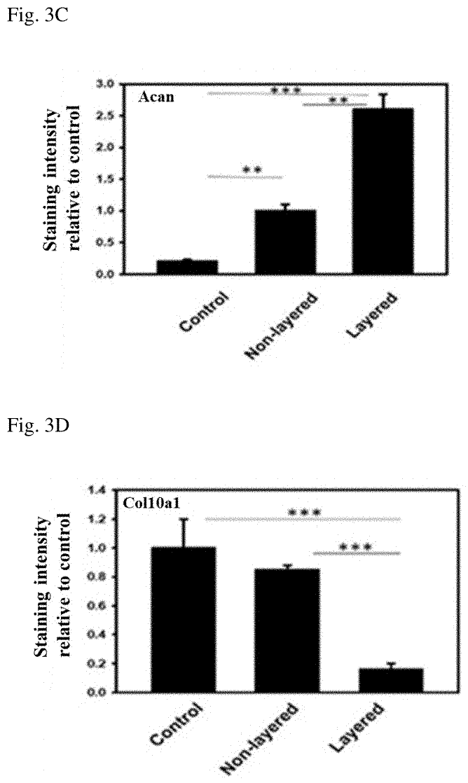

Articular joint samples at 12 weeks after surgery were harvested for gross and histologic evaluation. Mean gross grading was performed based on degree of defect coverage, neocartilage color, integration of the border zone, and surface smoothness. Twelve weeks after operation, the osteochondral defects regeneration was better in the groups implanted with heterogeneous ACs sheets (non-layered sheets) and stratified ACs sheets (layered sheets) than in controls (FIG. 2A). Grossly, implants of the layered sheets, the defects were completely covered with reparative tissue, whereas the osteochondral defects in the other 2 groups were partially filled (FIG. 2A). In addition, the newly formed tissue almost integrated with adjacent normal tissues in the group of layered sheets, and boundaries between implanted and native tissues were unclear than in the non-layered groups (FIG. 2A). Further, the articular surfaces were more intact, smooth, and resembling normal articular tissue in the group of layered sheets than in the non-layered groups (FIG. 2A). Quantitatively, the ICRS macroscopic scores for the non-layered group (9±0.3) and layered group (10.5±0.1) were obviously higher than that for controls (4±0.5) (FIG. 2B). Additionally, the score of layered ACs sheets treated defects was significantly higher than that in the non-layered ACs sheets treated defects (p<0.05) (FIG. 2B). To observe the cellular structure and matrix composition between the different implantation groups, the present invention performed microscopic histology with H&E (FIG. 2C), Safranin-O (FIG. 2D) and Alcian blue staining (FIG. 2E). Defects in the control groups contained a less cellular distribution and surrounded by loose connective tissue resembling fibrous tissue and staining very weakly for Alcian blue and Safranin-O. In contrast, defects implanted with non-layered sheets that most of the chondrocytes in the repair area were more evenly distributed in repair area than in the control groups and exhibited more Alcian blue and Safranin-O staining intensity. Further, the layered sheets implanted groups, the neocartilage displayed the zonal structure which was more closely resembled the native cartilage, in the superficial zone, cells were densely distributed and proteoglycan content (as stained by Safranin 0) was lower. In the middle zone, the proteoglycan content increased with depth and chondrocytes were rounded and more rarely populated than those in the superficial zone. Also, lacunae (empty triangles) were clearly observed. In the deep zone, chondrocytes were arranged in columns and cartilage-specific lacunae (solid triangles) presence. And also demonstrated the strongest Alcian blue and Safranin-0 staining intensity among the three groups. Finally, relative-quantitative evaluation of the quality of cartilage tissue regeneration using the Mankin histologic scoring system were 13.2, 4.3, and 1.8 for the control group, non-layered sheets group, and layered sheets group, respectively. That revealed a significantly improved histologic score in the layered sheets-treated defects compared with defects with no implant or non-layered sheets (FIG. 2F). To further characterize the composition of the neocartilage, the present invention performed IHC for Col-2a1, Aggrecan and Col-10a1 to detect mature cartilage matrix and hypertrophic cartilage matrix, respectively. As shown in FIG. 3A, the Col-2a1 and Aggrecan content of neocartilage in the non-layered and layered groups were positive staining but negatively in the control group. Further, the Col-2a1 was expressed in the extracellular matrix in the regenerated tissue within the defect. On the contrary, the Aggrecan remained both intracellularly localized and deposited into the extracellular matrix. In addition, the layered group presented more staining of Col-2a1 and Aggrecan than the non-layered group, which was in accordance with the results of IOD (integrated optical density) measurement (FIGS. 3B and 3C). Instead, these neocartilages stained positive for Col-10a1 in the control and non-layered groups (FIGS. 3A and 3D), indicating that they were fibrocartilages. In contrast, the layered group neocartilages were hyaline cartilages, as evidenced by the deposition of abundant proteoglycans and Col-2a1, and the absence of Col-10a1.

Those skilled in the art recognize the foregoing outline as a description of the method for communicating hosted application information. The skilled artisan will recognize that these are illustrative only and that many equivalents are possible.

What is claimed is:

1. A method for preparing zonal layered chondrocyte sheets, comprising the steps:
(a) providing a cartilage sample from a subject;
(b) isolating chondrocytes from the cartilage sample and then isolating superficial zone chondrocytes, middle zone chondrocytes and deep zone chondrocytes from the chondrocytes;
(c) seeding the deep zone chondrocytes in a culture medium in a culture dish and culturing the deep zone chondrocytes until reaching 90-100% cell confluence to form a deep zone chondrocyte sheet;
(d) seeding the middle zone chondrocytes on the top of the cultured deep zone chondrocyte sheet from the step (c) and culturing the middle zone chondrocytes until reaching 90-100% cell confluence to form a middle zone chondrocyte sheet; and
(e) seeding the superficial zone chondrocytes on the top of the cultured middle zone chondrocyte sheet from the step (d) and culturing the superficial zone chondrocytes until reaching 90-100% cell confluence to form a superficial zone chondrocyte sheet for obtaining the zonal layered chondrocyte sheets having the deep zone chondrocyte sheet, the middle zone chondrocyte sheet and the superficial zone chondrocyte sheet.

2. The method of claim 1, wherein the cartilage sample is an articular cartilage sample.

3. The method of claim 1, wherein the isolating method in the step (b) comprises using a technique of cell separation by density gradient centrifugation.

4. The method of claim 1, wherein the cell density of the superficial zone chondrocytes, the middle zone chondrocytes and the deep zone chondrocytes for seeding ranges from 1×10⁴ to 5×10⁴ cells/cm².

5. The method of claim 1, wherein the culture time of the zonal layered chondrocyte sheets after seeding the superficial zone chondrocytes in the step (e) ranges from 1 to 3 weeks.

6. The method of claim 1, wherein the culture medium for culturing the zonal layered chondrocyte in the step (e) comprises suramin.

7. A method for treating cartilage defects comprising administering a composition to a cartilage defect site of a subject, wherein the composition comprises zonal layered chondrocyte sheets prepared by the method of claim 1.

8. The method of claim 7, wherein the cartilage defects comprise articular cartilage defects.

9. The method of claim 8, wherein the route of administration of the composition comprises intraarticular administration.

10. A composition comprises zonal layered chondrocyte sheets prepared by the method of claim 1.


SEQUENCE LISTING (34 sequences)

SEQ ID NO: 1 - DNA, 19 bp, Artificial Sequence
  col-2a1 forward primer
actcctggcacggatggtc

SEQ ID NO: 2 - DNA, 20 bp, Artificial Sequence
  col-2a1 reverse primer
ctttctcaccaacatcgccc

SEQ ID NO: 3 - DNA, 20 bp, Artificial Sequence
  aggrecan forward primer
cccaaccagcctgacaactt

SEQ ID NO: 4 - DNA, 21 bp, Artificial Sequence
  aggrecan reverse primer
ccttctcgtgccagatcatca

SEQ ID NO: 5 - DNA, 25 bp, Artificial Sequence
  col-10a1 forward primer
tgaacttggttcatggagtgtttta

SEQ ID NO: 6 - DNA, 19 bp, Artificial Sequence
  col-10a1 reverse primer
tgccttggtgttggatggt

SEQ ID NO: 7 - DNA, 20 bp, Artificial Sequence
  gapdh forward primer
tcacgaccatggagaaggct

SEQ ID NO: 8 - DNA, 21 bp, Artificial Sequence
  gapdh reverse primer
caggaggcattgctgatgatc

SEQ ID NO: 9 - DNA, 22 bp, Artificial Sequence
  col-1a1 forward primer
ctggtacggcgagagcatgacc

SEQ ID NO: 10 - DNA, 22 bp, Artificial Sequence
  col-1a1 reverse primer
ggaggagcagggccttcttgag

SEQ ID NO: 11 - DNA, 22 bp, Artificial Sequence
  sox5 forward primer
ggccaagcagcagcaagaacag

SEQ ID NO: 12 - DNA, 22 bp, Artificial Sequence
  sox5 reverse primer
agctgaagcctggaggaaggag

SEQ ID NO: 13 - DNA, 23 bp, Artificial Sequence
  sox6 forward primer
cagccctgtcagtctgcctaaca

SEQ ID NO: 14 - DNA, 25 bp, Artificial Sequence
  sox6 reverse primer
gcatcttccgagcctcctgaatagc

SEQ ID NO: 15 - DNA, 22 bp, Artificial Sequence
  sox9 forward primer
ggcaatcccagggtccaccaac

SEQ ID NO: 16 - DNA, 24 bp, Artificial Sequence
  sox9 reverse primer
tggtcgaactcgttgacgtcgaag

SEQ ID NO: 17 - DNA, 22 bp, Artificial Sequence
  mmp13 forward primer
acccaggagccctcatgtttcc

SEQ ID NO: 18 - DNA, 22 bp, Artificial Sequence
  mmp13 reverse primer
cagggtttctcctcggagactg

SEQ ID NO: 19 - DNA, 22 bp, Artificial Sequence
  runx2 forward primer
ccagaccagcagcactccatac

SEQ ID NO: 20 - DNA, 22 bp, Artificial Sequence
  runx2 reverse primer
gggaactgctgtggcttccatc

SEQ ID NO: 21 - DNA, 21 bp, Artificial Sequence
  prg4 forward primer
ctcccaaggagcagcttctac

SEQ ID NO: 22 - DNA, 22 bp, Artificial Sequence
  prg4 reverse primer
ggtggtgggagctggttccttg

SEQ ID NO: 23 - DNA, 16 bp, Artificial Sequence
  pcna forward  primer
gcgcctggtccagggc

SEQ ID NO: 24 - DNA, 21 bp, Artificial Sequence
  pcna reverse primer
tcacgcccatggccaaattgc

SEQ ID NO: 25 - DNA, 20 bp, Artificial Sequence
  IL-1B forward primer
gtacatggttgctgcctgaa

SEQ ID NO: 26 - DNA, 20 bp, Artificial Sequence
  IL-1B reverse primer
ctagtgtgccatggtttcca

SEQ ID NO: 27 - DNA, 20 bp, Artificial Sequence
  IL-6 forward primer
ggcagaaaacaacctgaacc

SEQ ID NO: 28 - DNA, 20 bp, Artificial Sequence
  IL-6 reverse primer
gtggtggctttgtctggatt

SEQ ID NO: 29 - DNA, 20 bp, Artificial Sequence
  IL-8 forward primer
taggaccagagccaggaaga

SEQ ID NO: 30 - DNA, 20 bp, Artificial Sequence
  IL-8 reverse primer
cagtggggtccactctcaat

SEQ ID NO: 31 - DNA, 20 bp, Artificial Sequence
  TNFa forward primer
actgcacttcgaggttatcg

SEQ ID NO: 32 - DNA, 20 bp, Artificial Sequence
  TNFa reverse primer
gctggttgtctttcagcttc

SEQ ID NO: 33 - DNA, 20 bp, Artificial Sequence
  MIF forward primer
cgtgcgccctttgcagtctg

SEQ ID NO: 34 - DNA, 20 bp, Artificial Sequence
  MIF reverse primer
tggccgcgttcatgtcgtag